(19) United States
(12) Patent Application Publication
(10) Pub. No.: US 2026/0188523 A1
(43) Pub. Date: Jul. 2, 2026

(54) SYSTEMS AND METHODS FOR IMPROVING MACHINE RECOGNITION OF RELATIONSHIPS BETWEEN MEDICAL ENTITIES EXTRACTED FROM STRUCTURED RECORDS

(71) Applicant: Optum, Inc., Minnetonka, MN (US)

(72) Inventors: Ting-Yu Ho, Shoreline, WA (US); Guohua Zhao, Princeton, NJ (US); Feili Yu, Shoreline, WA (US)

(21) Appl. No.: 19/002,074

(22) Filed: Dec. 26, 2024

(51) Int. Cl. G16H 50/70; G06N 5/022; G16H 15/00

(52) U.S. Cl. CPC G16H 50/70; G06N 5/022; G16H 15/00

(57) ABSTRACT

A computer-implemented method includes receiving, by one or more processors, a set of structured records containing clinical information associated with one or more patients; extracting, by the one or more processors, a set of medical entities from the clinical information; determining, by the one or more processors, relationship strengths between pairs of the medical entities; and configuring, by the one or more processors, a computer-readable memory with a knowledge graph data structure in which nodes of the knowledge graph data structure represent the medical entities and the nodes are related to one another in the knowledge graph data structure based on the relationship strengths.

### FIELD OF THE INVENTION

The present disclosure relates generally to health care systems and services and, more particularly, to generation of knowledge graphs based on medical entities contained in clinical information.

### BACKGROUND

Health care data from electronic health records (EHRs) and insurance claims can be used in a variety of health care applications. For example, these data may be used to develop machine-learned models to assist both health care providers and payors. However, to gain an understanding of the vast volume of health care data from EHRs and insurance claims may require a comprehensive and repetitive data analysis, which can be complex, labor-intensive, and time consuming. Moreover, while various medical entities, such as medications, diagnoses, procedures, and the like may appear in an EHR, it may be difficult to determine how these various medical entities are related as the relationships may be vague based on the information contained in an EHR.

### SUMMARY

According to some embodiments of the disclosure, a computer-implemented method comprises: receiving, by one or more processors, a set of structured records containing clinical information associated with one or more patients; extracting, by the one or more processors, a set of medical entities from the clinical information; determining, by the one or more processors, relationship strengths between pairs of the medical entities; and configuring, by the one or more processors, a computer-readable memory with a knowledge graph data structure in which nodes of the knowledge graph data structure represent the medical entities and the nodes are related to one another in the knowledge graph data structure based on the relationship strengths.

In other embodiments, the set of structured records comprises a set of health care claims.

In still other embodiments, the set of health care claims comprises a provider claim and a pharmacy claim.

In still other embodiments, the set of health care claims is a first set of health care claims, the method further comprising: denoising, by the one or more processors, the first set of health care claims to generate a second set of health care claims that is a subset of the first set of health care claims; wherein extracting the data set of medical entities comprises: extracting, by the one or more processors, the data set of medical entities from the clinical information contained in the second set of health care claims.

In still other embodiments, determining the relationship strengths comprises: determining, by the one or more processors and for each of the pairs of the medical entities, a first conditional probability that a first entity in the pair is true given a second entity in the pair is true and a second conditional probability that the second entity in the pair is true given that the first entity in the pair is true.

In still other embodiments, determining the relationship strengths between the pairs of the medical entities comprises: performing a Bayesian statistical analysis of the set of medical entities from the clinical information; and determining, by the one or more processors, the relationship strengths between the pairs of the medical entities based on the Bayesian statistical analysis.

In still other embodiments, the medical entities comprise a diagnosis, a procedure, and a medication; and determining the relationship strengths comprises: determining, by the one or more processors, a first conditional probability that the diagnosis is true given the procedure is true; determining, by the one or more processors, a first conditional probability that the procedure is true given the diagnosis is true; determining, by the one or more processors, a second conditional probability that the diagnosis is true given the medication is true; determining, by the one or more processors, a first conditional probability that the medication is true given the diagnosis is true; determining, by the one or more processors, a second conditional probability that the procedure is true given the medication is true; and determining, by the one or more processors, a second conditional probability that the medication is true given the procedure is true.

In still other embodiments, extracting the set of medical entities from the clinical information further comprises: extracting, by the one or more processors, metadata from the clinical information that links the diagnosis to the procedure; and determining the first conditional probability that the diagnosis is true given the procedure is true comprises: determining, by the one or more processors, the first conditional probability that the diagnosis is true given the procedure is true based on the metadata.

In still other embodiments, the computer-implemented method further comprises: determining, by the one or more processors, a subset of medications in the set of medical entities having a common proprietary name; determining, by the one or more processors, medical substance names associated with the subset of medications having the common proprietary name; and determining, by the one or more processors, one or more product National Drug Codes (NDCs) that each have at least of the medical substance names; wherein the medication is representative of all of the one or more medications corresponding to the one or more product NDCs.

In still other embodiments, determining the one or more product National Drug Codes (NDCs) comprises: accessing, by the one or more processors, a database that associates NDCs with medical substance names; wherein the database comprises a Center for Disease Control (CDC) and Prevention database or a Federal Drug Administration (FDA) database.

In still other embodiments, the diagnosis comprises an International Classification of Diseases (ICD) code; the procedure comprises a Current Procedural Terminology (CPT) code or a Healthcare Common Procedure Coding System (HCPCS) code; and the medication comprises a National Drug Code (NDC).

In still other embodiments, the computer-implemented method further comprises: validating, by the one or more processors, an inference output from a machine-learned model using the knowledge graph data structure.

In still other embodiments, the machine-learned model is configured to extract medical information from an electronic health record.

In still other embodiments, the machine-learned model is configured to recommend a medical code for clinical information contained in an electronic health record; wherein the medical code is an International Classification of Diseases (ICD) code, Current Procedural Terminology (CPT) code, or a Healthcare Common Procedure Coding System (HCPCS) code.

According to some embodiments of the disclosure, a system comprises one or more processors; and one or more memories storing processor-executable instructions that, when executed by the one or more processors, cause the one or more processors to perform operations comprising: receiving a set of structured records containing clinical information associated with one or more patients; extracting a set of medical entities from the clinical information; determining relationship strengths between pairs of the medical entities; and configuring a computer-readable memory with a knowledge graph data structure in which nodes of the knowledge graph data structure represent the medical entities and the nodes are related to one another in the knowledge graph data structure based on the relationship strengths.

In further embodiments, the medical entities comprise a diagnosis, a procedure, and a medication; and determining the relationship strengths comprises: determining a first conditional probability that the diagnosis is true given the procedure is true; determining a first conditional probability that the procedure is true given the diagnosis is true; determining a second conditional probability that the diagnosis is true given the medication is true; determining a first conditional probability that the medication is true given the diagnosis is true; determining a second conditional probability that the procedure is true given the medication is true; and determining a second conditional probability that the medication is true given the procedure is true.

In still further embodiments, the operations further comprise: determining a subset of medications in the set of medical entities having a common proprietary name; determining medical substance names associated with the subset of medications having the common proprietary name; determining one or more product National Drug Codes (NDCs) that each have at least of the medical substance names; wherein the medication is representative of all of the one or more medications corresponding to the one or more product NDCs.

In still further embodiments, determining the one or more product National Drug Codes (NDCs) comprises: accessing a database that associates NDCs with medical substance names; wherein the database comprises a Center for Disease Control (CDC) and Prevention database or a Federal Drug Administration (FDA) database.

In still further embodiments, the operations further comprise: validating an inference output from a machine-learned model using the knowledge graph data structure.

According to some embodiments of the disclosure, one or more non-transitory computer-readable media store processor-executable instructions that, when executed by one or more processors, cause the one or more processors to perform operations comprising: receiving a set of structured records containing clinical information associated with one or more patients; extracting a set of medical entities from the clinical information; determining relationship strengths between pairs of the medical entities; and configuring a computer-readable memory with a knowledge graph data structure in which nodes of the knowledge graph data structure represent the medical entities and the nodes are related to one another in the knowledge graph data structure based on the relationship strengths.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the disclosure will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a system for associating medical entities, which are extracted from a set of structured records, in a knowledge graph in accordance with some embodiments of the disclosure;

FIG. 2 is a block diagram of the system for associating medical entities in a knowledge graph in accordance with some embodiments of the disclosure;

FIGS. 3 and 4 are flowcharts that illustrate operations of a system for associating medical entities, which are extracted from a set of structured records, in a knowledge graph in accordance with some embodiments of the disclosure;

FIG. 5 is a block diagram that illustrates validation of a machine-learned system through use of a knowledge graph in accordance with some embodiments of the disclosure;

FIG. 6 is a diagram that illustrates the relationships between two nodes in a knowledge graph;

FIG. 7 is a diagram that illustrates the relationships between medical entities in a knowledge graph in accordance with some embodiments of the disclosure;

FIGS. 8A and 8B are examples of structured records including errant information in accordance with some embodiments of the disclosure;

FIG. 9A is an example of aggregating multiple names for a medication for representation in a knowledge graph in accordance with some embodiments of the disclosure;

FIG. 9B is an example of conditional probabilities determined for various diagnoses for the medication of FIG. 9A;

FIG. 10 is a data processing system that may be used to implement a system for associating medical entities, which are extracted from a set of structured records, in a knowledge graph in accordance with some embodiments of the disclosure in accordance with some embodiments of the disclosure; and

FIG. 11 is a block diagram that illustrates a software/hardware architecture for use in a system for associating medical entities, which are extracted from a set of structured records, in a knowledge graph in accordance with some embodiments of the disclosure.

### DETAILED DESCRIPTION

In the following detailed description, numerous specific details are set forth to provide a thorough understanding of embodiments of the disclosure. However, it will be understood by those skilled in the art that embodiments of the disclosure may be practiced without these specific details. In some instances, well-known methods, procedures, components, and circuits have not been described in detail so as not to obscure the disclosure. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care products and/or services to a patient.

As used herein a “procedure” may be, but is not limited to, any type of treatment provided by a provider to a patient or any type of medicine or product prescribed or given to a patient for treatment. In general, a “procedure” may be defined as any activity directed at or performed on an individual with the object of improving health, treating disease or injury, or making a diagnosis.

As used herein a medical entity may include, but is not limited to a disease (e.g., medical conditions and diagnoses), a medication (e.g., pharmaceutical drugs or drug therapies used for treatment), a procedure (e.g., diagnostic procedures, therapeutic procedures, and medical devices), lab (e.g., pathology and laboratory procedures), and a symptom (e.g., clinical signs and symptoms). Within the medication category, the following sub-categories may be used: a dosage (e.g., amount or strength of a drug including units), form (e.g., the physical form of a dose of drug when it is administered), and a route (e.g., the way in which a drug is taken into the body).

Embodiments of the disclosure are described herein in the context of a system for associating medical entities, which are extracted from a set of structured records, in a knowledge graph. The knowledge graph may be used to validate or audit inferences made by one or more machine-learned models. The one or more machine-learned models may be embodied in a variety of different ways including, but not limited to, one or more of the following AI systems: a multi-layer neural network, a machine learning system, a deep learning system, a large language model, a natural language processing system, and/or computer vision system. Moreover, it will be understood that the multi-layer neural network is a multi-layer artificial neural network comprising artificial neurons or nodes and does not include a biological neural network comprising real biological neurons. The machine-learned models described herein may be configured to transform a memory of a computer system to include one or more data structures, such as, but not limited to, arrays, extensible arrays, linked lists, binary trees, balanced trees, heaps, stacks, and/or queues. These data structures can be configured or modified through the adjudication process and/or the machine learning training process to improve the efficiency of a computer system when the computer system operates in an inference mode to make an inference, prediction, classification, suggestion, or the like with respect to medical entities.

Significant and valuable insight may be obtained from health care data contained in electronic health records (EHRs) and insurance claims. One way to organize this information is through use of a knowledge graph in which the nodes represent medical entities, and the edges represent the relationships between the various entities. Construction of a knowledge graph for a health care application, however, may be challenging due to a lack of a representative knowledge graph construction taxonomy. Example health care related knowledge graphs may be built by humans with domain knowledge of health care, but such a build approach may be slow and costly. Attempts to build a health care knowledge graph based on electronic health records associated with patients have been met with challenges due to the lack of a definitive mapping between clinical medical entities, such as drugs, diagnoses, procedures, and the like. For example, even though Drug A and Drug B appear in the same electronic health record, it may not be clear how these two drugs are related. Also, the appearance of Drug A and Disease C in an electronic health record does not necessarily mean that Drug A is being used to treat Disease C. A patient's chart or health record often includes multiple types of symptoms, diagnoses, and drugs. These vague relationships may make it difficult to build knowledge graphs from electronic health records.

Advantageously, some embodiments of the disclosure stem from a realization that structured records, such as patient medical claims, contain clinical information related to medical entities in a structured format that is amenable to determining the relationship between these medical entities. Some embodiments of the disclosure may provide a system and method for receiving a set of structured records, such as medical claims that contain clinical information associated with one or more patients and extracting a set of medical entities from the clinical information. Relationship strengths between the pairs of medical entities may be determined using, for example, a Bayesian statistical analysis of the medical entities in the set of structured records. The Bayesian statistical analysis may provide conditional probabilities for pairs of first and second medical entities. For example, the Bayesian statistical analysis provides a determination of the conditional probability that a first medical entity is present or “true” when it is assumed or given that a second medical entity is present or “true.” These relationship strengths between the medical entities, which may be conditional probabilities in some embodiments, may be used as edges in a knowledge graph in which the nodes correspond to the extracted medical entities. In some embodiments, the medical entities may comprise diagnoses, procedures, and medications. A computer-readable memory may be configured with the knowledge graph data structure in which the nodes are the extracted medical entities, and the edges are the relationship strengths. Advantageously, in some embodiments, the knowledge graph data structure may be used to validate or audit the inferences generated by machine-learned models, such as machine-learned models that are configured to generate an inference output based on extracted medical information from an EHR, such as a proposed procedure, medication, diagnosis validation, or the like. The knowledge graph data structure may also be used to validate or audit the inferences generated by machine-learned models used in recommending medical codes for clinical information contained in an EHR.

Advantageously, one or more of the medical entities used in the knowledge graph data structure may be selected to have different quantization levels, e.g., 64-bit, 32-bit, etc. to reduce the size of the knowledge graph data structure that is stored in the computer-readable memory. For example, the quantization level for representing various medications, diagnoses, or procedures may be varied depending on a degree of precision desired for distinguishing between various ones of these entities in the same category, i.e., distinguishing one medication from another or distinguishing between like entities, e.g., labeled and generic versions of the same medication.

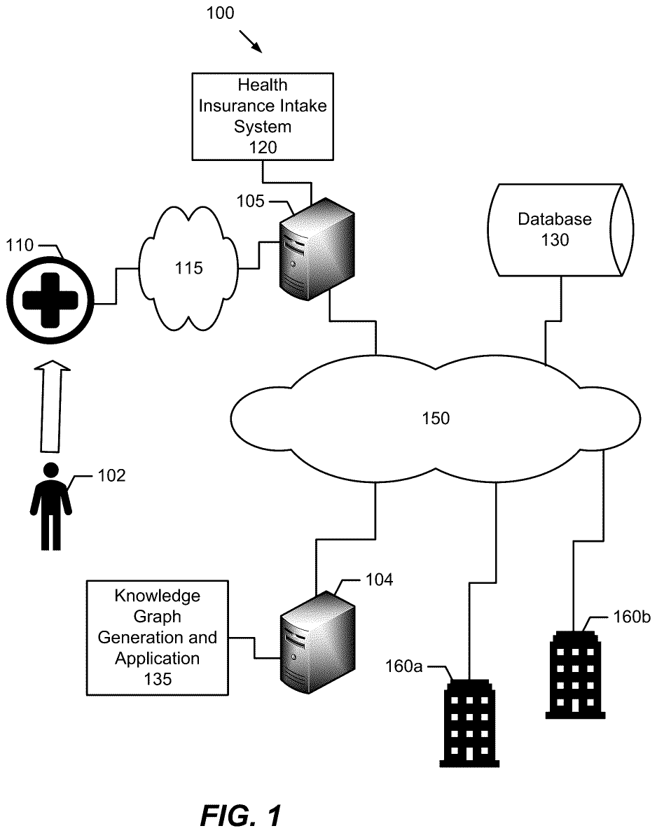

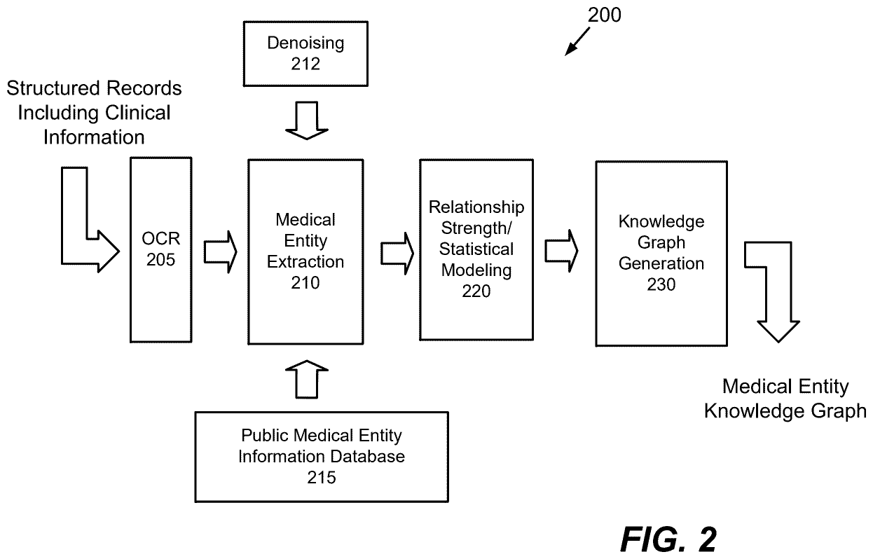

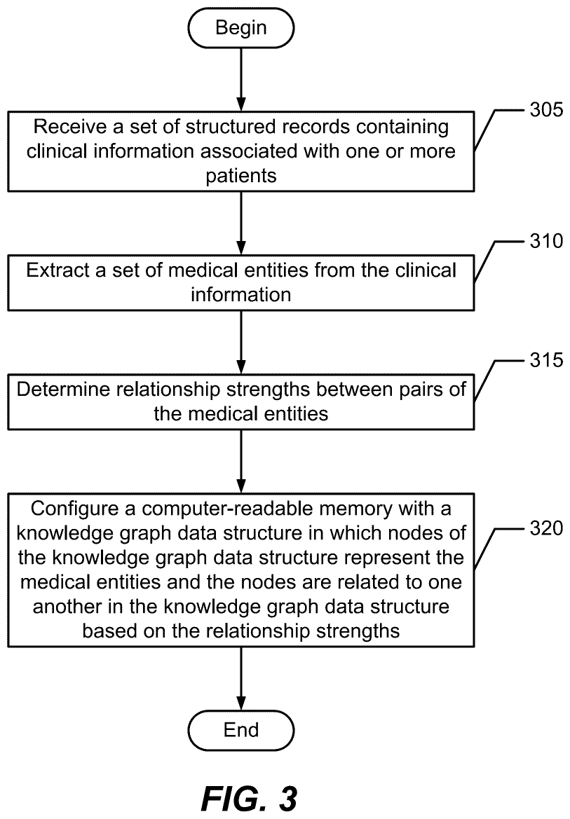

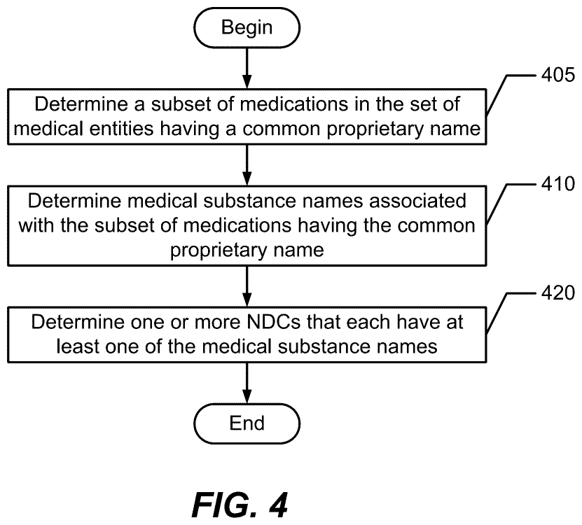

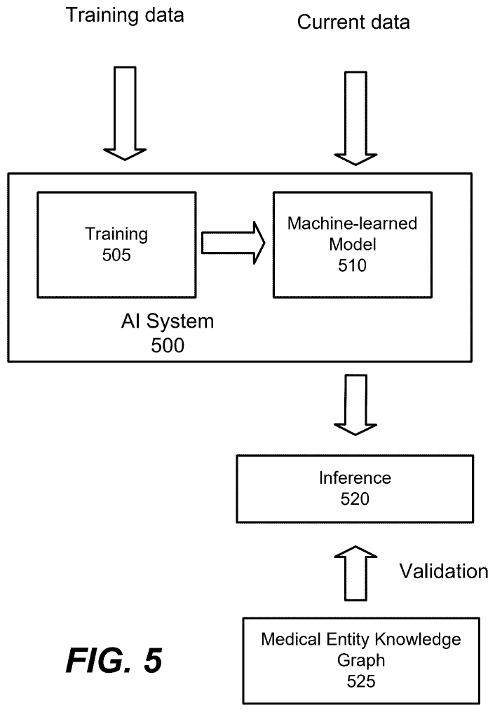

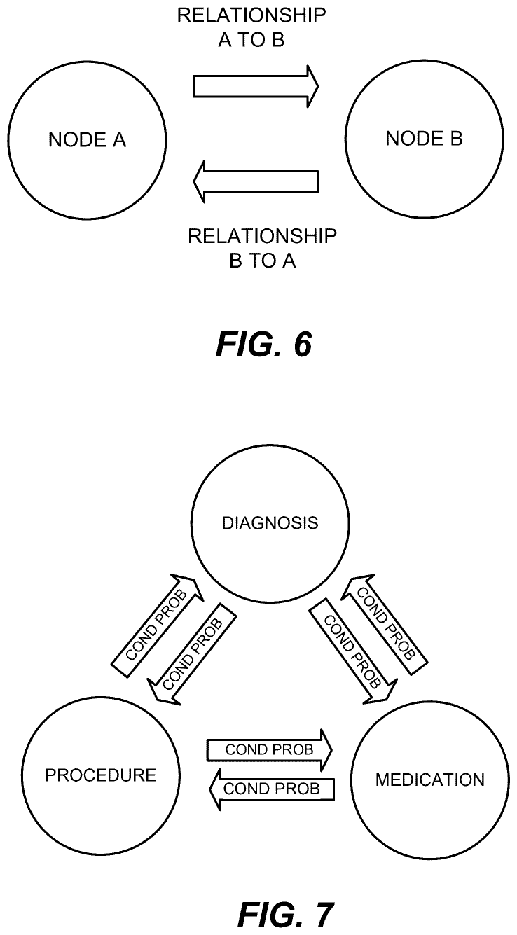

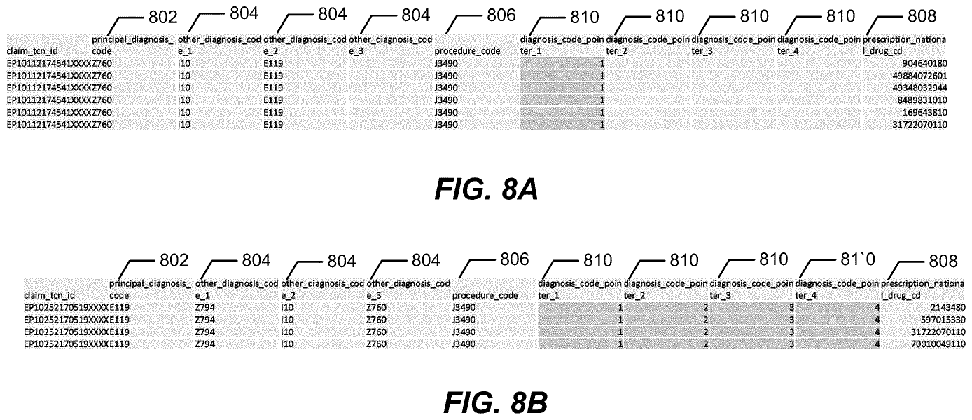

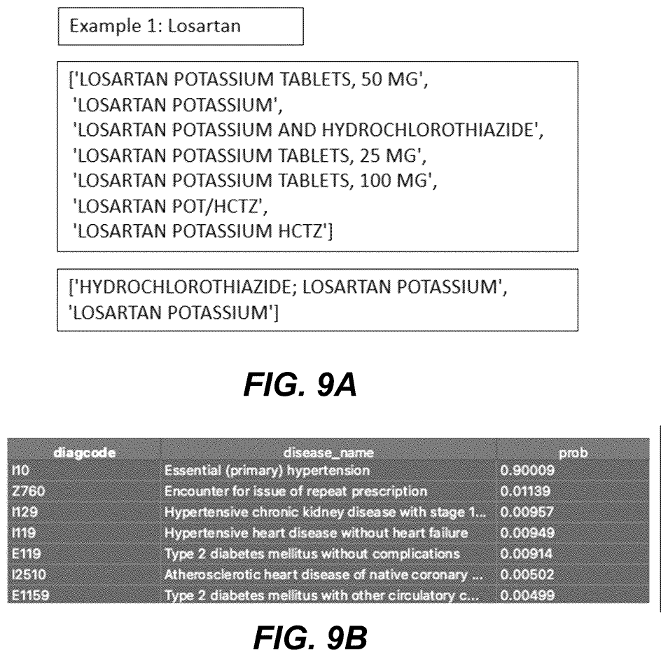

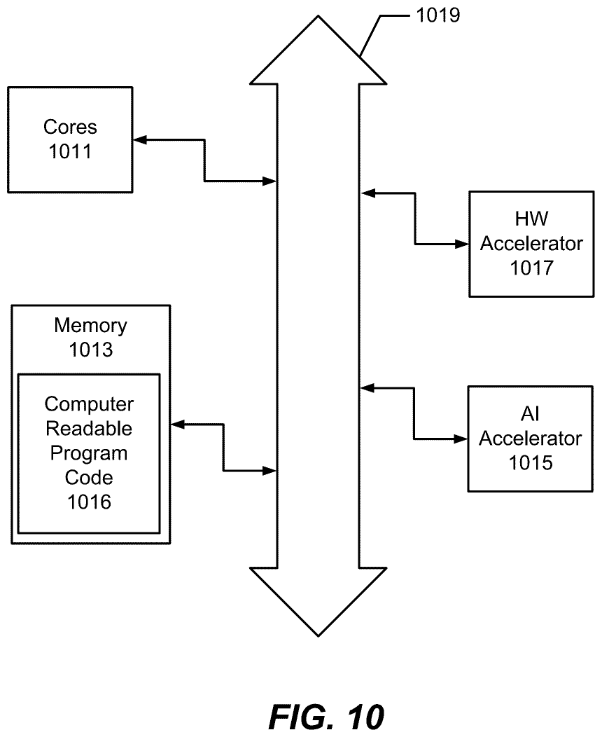

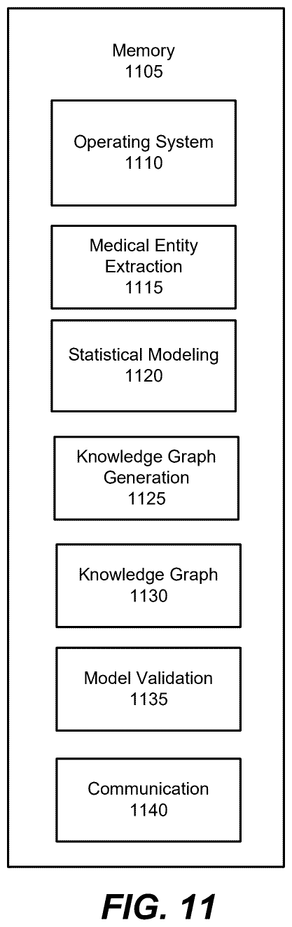

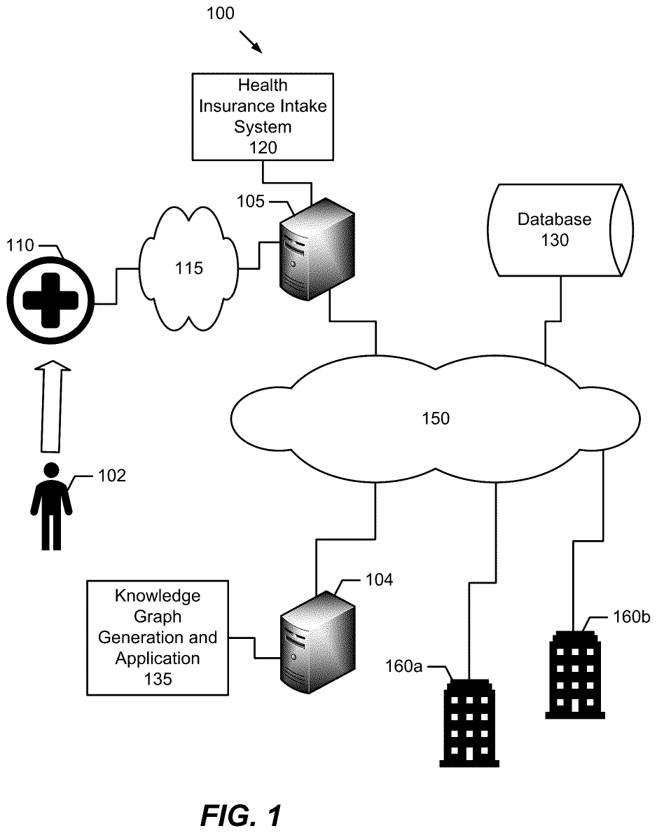

Referring to FIG. 1, a communication network 100 including a system for associating medical entities, which are extracted from a set of medical records, in a knowledge graph, in accordance with some embodiments of the disclosure, comprises one or more facilities 110, which may represent various types of organizations that are used to deliver health care services and products to patients 102. Organizations that are used to deliver health care services to patients via health care professionals are referred to generally herein as “providers.” The providers may include, but are not limited to, hospitals, medical practices, mobile patient care facilities, diagnostic centers, lab centers, pharmacies, and the like. The providers may operate by providing health care services for patients and then invoicing one or more payors 160a and 160b for the services rendered. The payors 160a and 160b may include, but are not limited to, providers of private insurance plans, providers of government insurance plans (e.g., Medicare, Medicaid, state, or federal public employee insurance plans), providers of hybrid insurance plans (e.g., Affordable Care Act plans), providers of private medical cost sharing plans, and the patients themselves. One provider facility 110 is illustrated in FIG. 1 with the provider including a patient intake/accounting system server 105 accessible via a network 115. The patient intake/accounting system server 105 may be configured with a health insurance intake system module 120 to manage the intake of patients for appointments and to generate invoices for payors, e.g., in the form of insurance claims, for services and products rendered through the provider 110. The network 115 communicatively couples the patient intake/accounting system server 105 to other devices, terminals, and systems in the provider's facility 110. The network 115 may comprise one or more local or wireless networks to communicate with patient intake/accounting system server 105 when the patient intake/accounting system server 105 is located in or proximate to the service provider facility 110. When the patient intake/accounting system server 105 is in a remote location from the health care facility, such as part of a cloud computing system or at a central computing center, then the network 115 may include one or more wide area or global networks, such as the Internet.

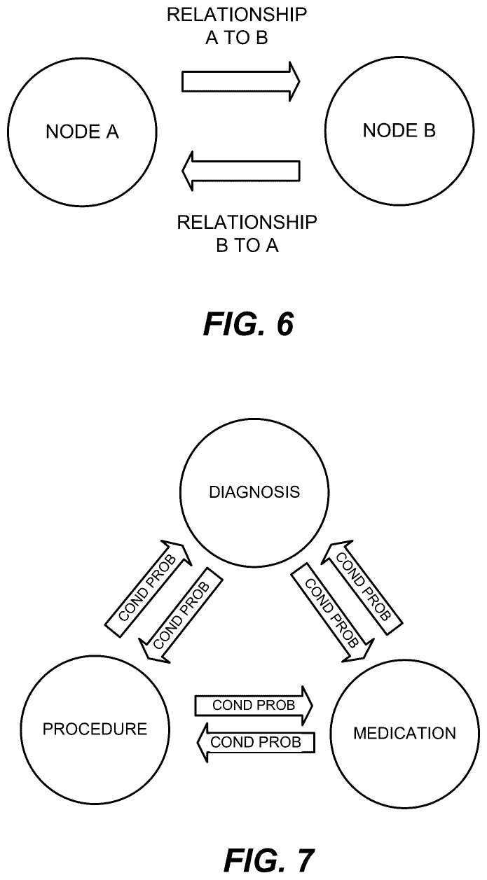

According to some embodiments of the disclosure, a medical entity knowledge graph server 104 may include a knowledge graph generation and application module 135 that is configured to be an intermediary system between the provider facilities 110 and the payors 160a, 160b and to access the structured records, e.g., claims generated by the patient intake/accounting system server 105. The knowledge graph generation and application module 135 may be configured to extract medical entities and to generate a knowledge graph data structure that associates the extracted medical entities with one another. As shown in FIG. 6, a knowledge graph is a semantic network that visualizes entities and the relationships between them. The information represented by the knowledge graph may be stored in a graph database or data structure. An entity is an object, such as an event, person, or thing. In a knowledge graph, these entities are represented as nodes. Each node/entity may be related to other nodes/entities. The relationships are represented by edges, which are connections between the nodes. In extracting and determining the entities used in the knowledge graph data structure, the knowledge graph generation and application module 135 may be configured to access one or more external databases 130, such as databases provided by the Center for Disease Control (CDC) and Prevention and/or the Federal Drug Administration (FDA). The information contained in these databases may be used to determine National Drug Codes (NDCs) for medications based on substance names or proprietary names, for example, or medication names (substance or proprietary name) based on NDCs.

The knowledge graph generation and application module 135 may be further configured to use the knowledge graph data structure to validate or audit the inferences generated by machine-learned models, such as machine-learned models that are configured to generate an inference output based on extracted medical information from an EHR, such as a proposed procedure, medication, diagnosis validation, or the like. The knowledge graph data structure may also be used to validate or audit the inferences generated by machine-learned models used in recommending medical codes for clinical information contained in an EHR.

A network 150 couples the patient intake/accounting system server 105 and payors 160a, 160b to the medical entity knowledge graph server 104. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The system for associating medical entities, which are extracted from a set of medical records, in a knowledge graph, provided through the medical entity knowledge graph server 104 may, in some embodiments, be embodied as a cloud service. For example, health care service providers 110 and/or payors 160a, 160b may access the medical entity knowledge graph service as a Web service. In some embodiments, the medical entity knowledge graph service may be implemented as a Representational State Transfer Web Service (RESTful Web service).

Although FIG. 1 illustrates an example communication network including a system for associating medical entities, which are extracted from a set of medical records, in a knowledge graph, in accordance with some embodiments of the disclosure, it will be understood that embodiments of the inventive subject matter are not limited to such configurations, but are intended to encompass any configuration capable of carrying out the operations described herein.

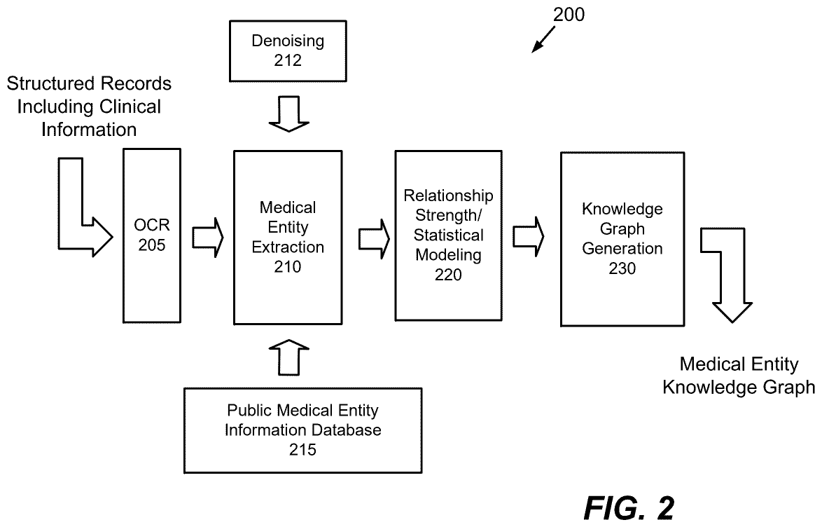

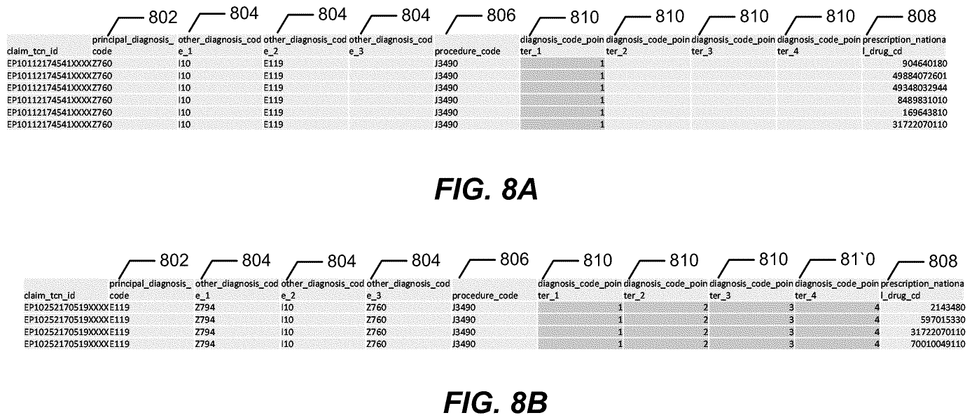

FIG. 2 is a block diagram illustrating a system 200 for associating medical entities in a knowledge graph that may be used in the medical entity knowledge graph server 104 and the knowledge graph generation and application module 135 in accordance with some embodiments of the disclosure. As shown in FIG. 2, the multi-stage system 200 for associating medical entities in a knowledge graph includes a plurality of modules coupled in pipeline fashion. The multi-stage system 200 for associating medical entities in a knowledge graph may be configured automate the operations involved in extracting medical entities from a set of structured records, such as health care claims, and associating the medical entities with one another as pairings in a knowledge graph that can be used to configure a non-transitory computer readable memory. The multi-stage system 200 for associating medical entities in a knowledge graph includes the following serially connected modules: an Optical Character Recognition (OCR) module 205, a medical entity extraction module 210, a relationship strength/statistical modeling module 220, and a knowledge graph generation module 230. The OCT module 205 is configured to convert the structured records, such as patient medical and/or pharmacy claim records, that include clinical information into text records. The medical entity extraction module 210 is configured to extract medical entities from clinical information included in the set of structured records, e.g., medical and/or pharmacy claim records. In some embodiments, the medical entity categories may be procedure, diagnosis, and medication. A diagnosis may be identified based on an International Classification of Diseases (ICD) code. A procedure may be identified by a Current Procedural Terminology (CPT) code and/or a Healthcare Common Procedure Coding System (HCPCS) code. A medication may be identified using a National Drug Code (NDC). Some of the structured records, however, may be discarded as including errant information. The denoising module 212 may be configured to reduce the set of structured records to a subset thereof by discarding one or more of the structured records. As shown in FIGS. 8A and 8B, a health care claim may have a principal diagnosis code 802 and one or more additional diagnosis codes labeled as “other” diagnosis codes 804. A medical or pharmacy claim may also include one or more procedure codes 806 and one or more prescription or medication codes 808. To link diagnoses to procedures, the health care claim may include diagnosis code pointers 810 that identify that a particular diagnosis code is linked to a particular procedure. These pointers may be viewed as metadata that can be used in determining the conditional probability that if a particular procedure is present or “true” in the clinical data of a health care claim, then a particular diagnosis is also present or “true.” In the examples of FIGS. 8A and 8B, however, the diagnosis code pointers (first diagnosis code pointer in FIG. 8A and all four diagnosis code pointers in FIG. 8B) include the same number repeated across all procedure codes. This is clearly errant information and indicative that the clinical data in the claim may not be trustworthy. As a result, the denoising module 212 may discard these claims from the set of structured records. The public medical entity information database module 215 may be configured to access the database 130 to determine NDCs for medications based on substance names or proprietary names, for example, or medication names (substance or proprietary name) based on NDCs. The relationship strength/statistical modeling module 220 may be configured to determine relationship strengths between pairs of the medical entities that were extracted by the medical entity extraction module 210. Relationship strengths between the pairs of medical entities may be determined using, for example, a Bayesian statistical analysis of the medical entities in the set of structured records. The Bayesian statistical analysis may provide conditional probabilities for pairs of first and second medical entities as shown in FIG. 7. For example, the Bayesian statistical analysis provides a determination of the conditional probability that a first medical entity is present or “true” when it is assumed or given that a second medical entity is present or “true.” These relationship strengths between the medical entities, which may be conditional probabilities in some embodiments, may be used as edges in a knowledge graph in which the nodes correspond to the extracted medical entities. In some embodiments, the medical entities may comprise diagnoses, procedures, and medications. The knowledge graph generation module is configured to configure a non-transitory computer-readable memory with a knowledge graph data structure in which nodes of the knowledge graph data structure represent the medical entities and the nodes are related to each other based on the relationship strengths, which, in some embodiments, may be conditional probabilities as described above.

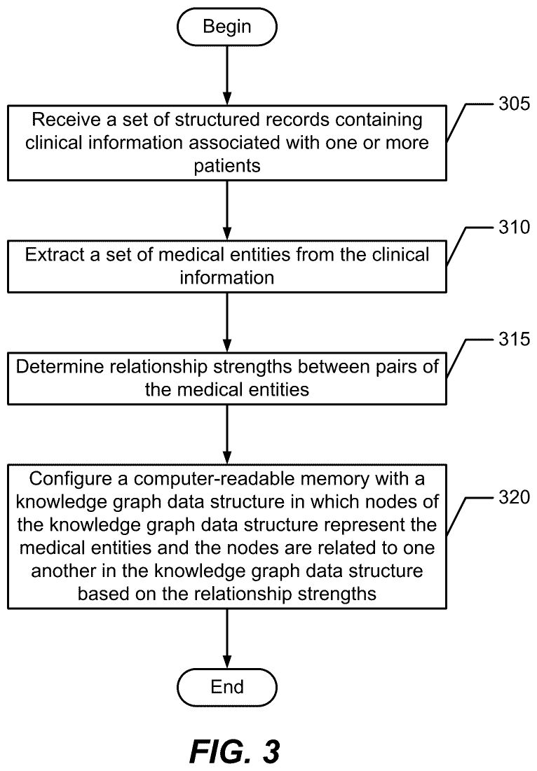

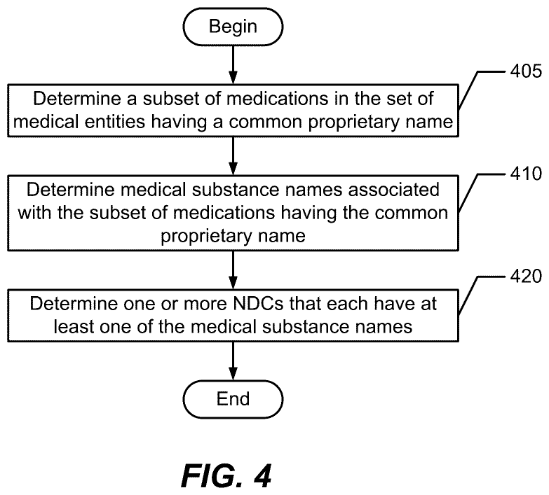

FIGS. 3 and 4 are flowcharts that illustrate operations of a system for associating medical entities, which are extracted from a set of structured records, in a knowledge graph in accordance with some embodiments of the disclosure. Operations begin at block 305 where a set of structured records are received that contain clinical information associated with one or more patients. A set of medical entities are extracted from the clinical information at block 310. At block 315 relationship strengths between pairs of the medical entities are determined. A non-transitory computer-readable memory is configured with a knowledge graph data structure in which nodes of the knowledge graph data structure represent the medical entities and the nodes are related to one another in the knowledge graph data structure based on the relationship strengths at block 320.

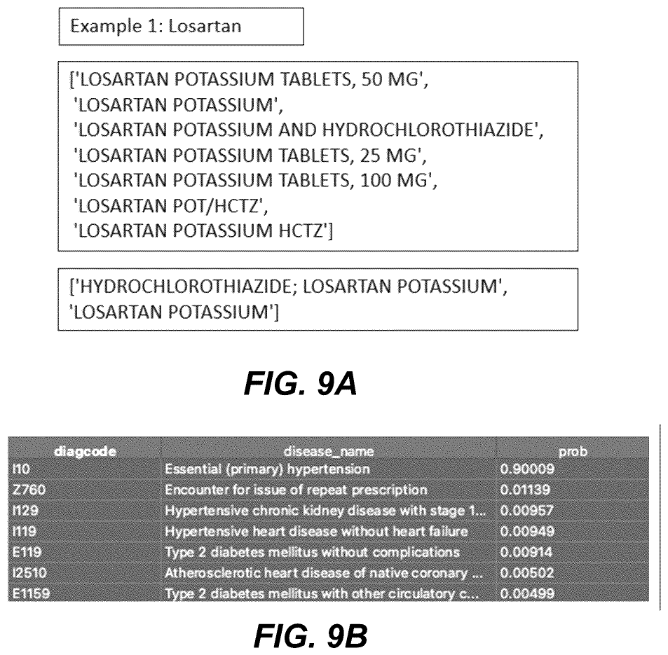

For many medications, there is the potential that what is essentially the same medication is described using different names. For example, a medication may have a labeled proprietary name and also a generic version name. Moreover, each high-level name may have different variations based on dosage levels or supplemental ingredients or substances included with the medication. It may be desirable to aggregate all of these different variations under the same medication name used as a node in the knowledge graph data structure. FIG. 9A illustrates an example for a mediation with the proprietary name Losartan. There are several different variations in how this medication may be described in a health care or pharmacy claim. FIG. 4 is a flowchart that illustrates example operations for aggregating the different instances of a medication under a common entity name, according to some embodiments of the disclosure. Operations begin at block 405 where a subset of medical entities having a common proprietary name are determined. At block 410, the medical substance names associated with the subset of medications having the common proprietary name are determined. One or more NDCs that each have at least one of the medical substance names is determined at block 420. The node in the knowledge graph that is representative of the medication associated with the common proprietary name may be representative of all the medications associated with the NDCs that have one or more of the medical substance names included in the medication having the proprietary name. Referring to the example of FIG. 9A with respect to the medication Losartan, after aggregating all of the medications that fall under the Losartan umbrella as described above with respect to flowchart of FIG. 4, there is a conditional probability of approximately 90% that if a patient is prescribed Losartan, the patient has essential (primary) hypertension corresponding to diagnosis code 110 as shown in FIG. 9B. The conditional probabilities that other diagnosis codes are implicated if a patient is prescribed Losartan are all very low (approximately 1% or less).

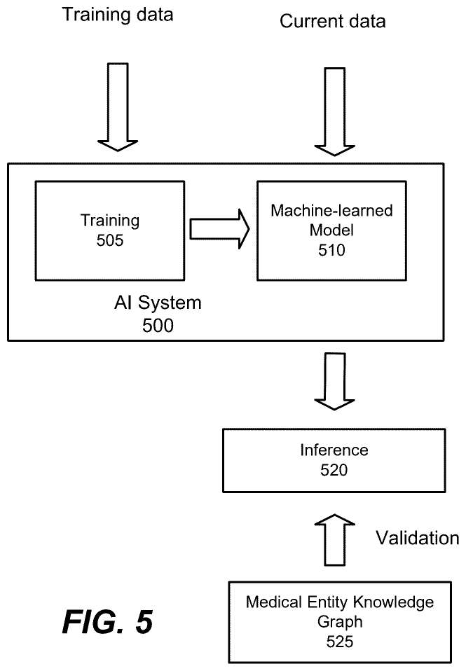

In some embodiments, the knowledge graph data structure may be used to validate or audit the inferences generated by machine-learned models. Referring now to FIG. 5, an AI system 500 may include a training module 505 and a machine-learned model 510. The training module 505 is configured to receive training data. The training process generates learned associations between features of the training data and inferences that may be drawn therefrom. Based on these learned associations a machine-learned model 510 is generated. When operating in inference mode, the machine-learned model 510 is applied to new or current data, which is of the same type as the training data to generate an inference 520 based on the current data. The training module 505 may perform the training and the machine-learned model 510 may operate in inference mode on a same set of one or more processors. In other embodiments, the object training module 505 may perform the training operations using one or more processors included in a first computing entity and the machine-learned model 510 may operate in inference mode using one or more processors included in a second computing entity different from the first computing entity. The medical entity knowledge graph data structure 525 may be used to valid or audit the inferences generated by the machine-learned model 510. For example, in some embodiments, the machine-learned model 510 may be configured to generate an inference output based on extracted medical information from an EHR, such as a proposed procedure, medication, diagnosis validation, or the like. If the inference 520 output from the machine-learned model 510 indicates a diagnosis of X, but the knowledge graph 525 indicates that there is a very low conditional probability of the diagnosis of X based on one or more procedures and/or one or more medications found in the EHR, then this may be indicative that the inference 520 needs to be questioned or evaluated. In other embodiments, the machine-learned model 510 may be configured to generate an inference output that is a recommended medical code for clinical information contained in the EHR. Again, if the inference 520 output from the machine-learned model 510 indicates a coding recommendation for a diagnosis of X, but the knowledge graph 525 indicates that there is a very low conditional probability of the diagnosis of X based on one or more procedures and/or one or more medications found in the EHR, then the coding recommendation may need to be questioned or evaluated.

In some embodiments, the features of the training data used during training of the machine-learned model 510 may be evaluated to determine their relative impact on the accuracy in generating the inference 520. One or more features of the training data having a lesser impact on the accuracy of the inference 520 than other features may be removed from the training data to reduce the overall dimensionality of the training data set used to train the machine-learned model. By sparsifying the features used during training and/or during inference mode of the machine-learned model 510, the model may execute on one or more processors with decreased memory and computational requirements (e.g., processor speed and availability).

In some embodiments in which the machine-learned model 510 comprises a neural network, one or more nodes or paths of the neural network may be removed through setting certain parameters or weights to zero based on the sparsification of the features used during training and during inference mode. As a result, the sparsified neural network may run more efficiently and use fewer memory resources.

In some embodiments, one or more of the features used in training or during inference mode of the machine-learned model 510 may be selected to have different quantization levels, e.g., 64-bit, 32-bit, etc. to reduce the size and complexity of the machine-learned model 510. The quantization level may be adjusted to achieve a desired inference accuracy rate.

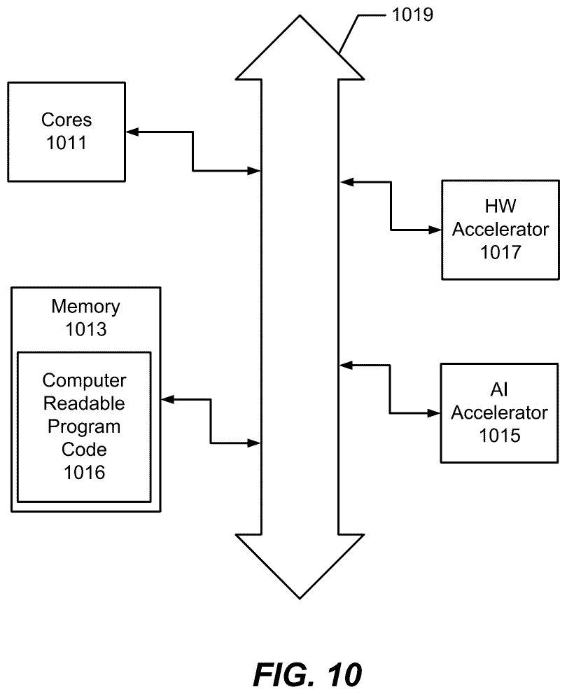

FIG. 10 is a data processing system that may be used to implement a system for associating medical entities, which are extracted from a set of structured records, in a knowledge graph in accordance with some embodiments of the disclosure in accordance with some embodiments of the disclosure. For example, the data processing system of FIG. 10 may be used to implement the medical entity knowledge graph server 104 of FIG. 1, the system 200 for associating medical entities in a knowledge graph of FIG. 2, and/or the AI system 500 of FIG. 5. As shown in FIG. 10, the data processing system may include at least one core 1011, a memory 1013, an artificial intelligence (AI) accelerator 1015, and a hardware (HW) accelerator 1017. The at least one core 1011, the memory 1013, the AI accelerator 1015, and the HW accelerator 1017 may communicate with each other through a bus 919.

The at least one core 1011 may be configured to execute computer program instructions. For example, the at least one core 1011 may execute an operating system and/or applications represented by the computer readable program code 1016 stored in the memory 1013. In some embodiments, the at least one core 1011 may be configured to instruct the AI accelerator 1015 and/or the HW accelerator 1017 to perform operations by executing the instructions and obtain results of the operations from the AI accelerator 1015 and/or the HW accelerator 1017. In some embodiments, the at least one core 1011 may be an Application Specific Instruction Set Processor (ASIP) customized for specific purposes and support a dedicated instruction set.

The memory 1013 may have an arbitrary structure configured to store data. For example, the memory 1013 may include a volatile memory device, such as dynamic random-access memory (DRAM) and static RAM (SRAM), or include a non-volatile memory device, such as flash memory and resistive RAM (RRAM). The at least one core 1011, the AI accelerator 1015, and the HW accelerator 1017 may store data in the memory 1013 or read data from the memory 1013 through the bus 1019.

The AI accelerator 1015 may refer to hardware designed for AI applications. In some embodiments, the AI accelerator 1015 may include a machine-learned model and/or other AI models configured to facilitate operations of associating medical entities, which are extracted from a set of structured records, in a knowledge graph as described above with respect to FIGS. 1-7, 8A, 8B, 9A, and 9B. The AI accelerator 1015 may generate output data by processing input data provided from the at least one core 1015 and/or the HW accelerator 1017 and provide the output data to the at least one core 1011 and/or the HW accelerator 1017. In some embodiments, the AI accelerator 1015 may be programmable and be programmed by the at least one core 1011 and/or the HW accelerator 1017. The HW accelerator 1017 may include hardware designed to perform specific operations at high speed. The HW accelerator 1017 may be programmable and be programmed by the at least one core 1011.

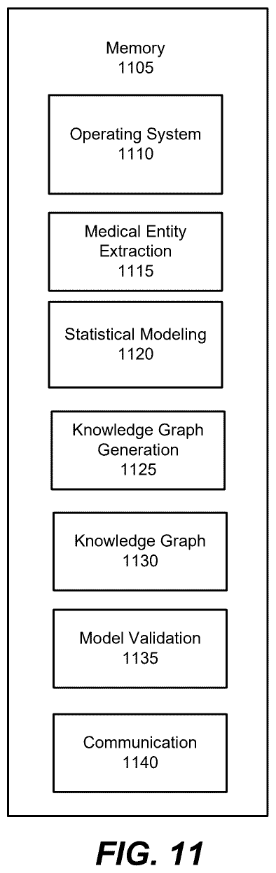

FIG. 11 illustrates a memory 1105 that may be used in embodiments of data processing systems, such as the medical entity knowledge graph server 104 of FIG. 1, the system 200 for associating medical entities in a knowledge graph of FIG. 2, the AI system 500 of FIG. 5, and/or the data processing system of FIG. 10 to facilitate operations of associating medical entities, which are extracted from a set of structured records, in a knowledge graph in accordance with some embodiments of the disclosure. The memory 1105 is representative of the one or more memory devices containing the software and data used for facilitating operations the medical entity knowledge graph server 104 of FIG. 1, the system 200 for associating medical entities in a knowledge graph of FIG. 2, and/or the AI system 500 of FIG. 5 as described herein. The memory 1105 may include, but is not limited to, the following types of devices: cache, ROM, PROM, EPROM, EEPROM, flash, SRAM, and DRAM. As shown in FIG. 11, the memory 1105 may contain five or more categories of software and/or data: an operating system 1110, a medical entity extraction module 1115, a statistical modeling module 1120, a knowledge graph generation module 1125, a knowledge graph 1130, and a model validation module 1135. In particular, the operating system 1110 may manage the data processing system's software and/or hardware resources and may coordinate execution of programs by the processor. The medical entity extraction module 1115 may be configured to perform one or more of the operations described above with respect to the medical entity extraction module 210 and the denoising module 212 of FIG. 2 and the flowcharts of FIGS. 3 and 4. The statistical modeling module 1120 may be configured to perform one or more of the operations described above with respect to relationship strength/statistical modeling module 220 of FIG. 2 and the flowcharts of FIGS. 3 and 4. The knowledge graph generation module 1125 may be configured to perform one or more of the operations described above with respect to the knowledge graph generation module 230 of FIG. 2 and the flowcharts of FIGS. 3 and 3. The knowledge graph 1130 may correspond to the medical entity knowledge graph of FIG. 2 and the medical entity knowledge graph 525 of FIG. 5. The model validation module 1135 may be configured to perform one or more of the operations described above with respect to the AI system 500 of FIG. 5. The communication module 1140 may be configured to facilitate communication between the facilities 110, the medical entity knowledge graph server 104, and the database 130 of FIG. 1, the system 200 for associating medical entities in a knowledge graph of FIG. 2, and/or the AI system 500 of FIG. 5.

Although FIG. 11 illustrates hardware/software architectures that may be used in data processing systems, such as the medical entity knowledge graph server 104 of FIG. 1, the system 200 for associating medical entities in a knowledge graph of FIG. 2, the AI system 500 of FIG. 5, and/or the data processing system of FIG. 10, respectively, in accordance with some embodiments of the disclosure, it will be understood that embodiments of the present disclosure are not limited to such a configuration but is intended to encompass any configuration capable of carrying out operations described herein.

Computer program code for carrying out operations of data processing systems discussed above with respect to FIGS. 1-11 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the medical entity knowledge graph server 104 of FIG. 1, the system 200 for associating medical entities in a knowledge graph of FIG. 2, the AI system 500 of FIG. 5, and/or the data processing system of FIG. 10 may each be implemented as a single processor system, a multi-processor system, a multi-core processor system, or even a network of stand-alone computer systems, in accordance with various embodiments of the disclosure. Each of these processor/computer systems may be referred to as a “processor” or “data processing system.” The functionality provided by each of the medical entity knowledge graph server 104 of FIG. 1, the system 200 for associating medical entities in a knowledge graph of FIG. 2, the AI system 500 of FIG. 5, and/or the data processing system of FIG. 10 may be embodied as a single server or embodied as separate servers in accordance with different embodiments of the disclosure.

The data processing apparatus described herein with respect to FIGS. 1-10 may be used to facilitate operations of associating medical entities, which are extracted from a set of structured records, in a knowledge graph in accordance with some embodiments of the disclosure. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 1013 and memory 1105 when coupled to a processor includes computer readable program code that, when executed by the processor, causes the processor to perform operations including one or more of the operations described herein with respect to FIGS. 1-7, 8A, 8B, 9A, and 9B.

Some embodiments of the disclosure may provide a system and a method for associating medical entities, which are extracted from a set of structured records, in a knowledge graph as set forth by the following examples: Example 1: a computer-implemented method comprises: receiving, by one or more processors, a set of structured records containing clinical information associated with one or more patients; extracting, by the one or more processors, a set of medical entities from the clinical information; determining, by the one or more processors, relationship strengths between pairs of the medical entities; and configuring, by the one or more processors, a computer-readable memory with a knowledge graph data structure in which nodes of the knowledge graph data structure represent the medical entities and the nodes are related to one another in the knowledge graph data structure based on the relationship strengths.

Example 2: the computer-implemented method of Example 1, wherein the set of structured records comprises a set of health care claims.

Example 3: the computer-implemented method of Example 2, wherein the set of health care claims comprises a provider claim and a pharmacy claim.

Example 4: the computer-implemented method of Examples 2-3, wherein the set of health care claims is a first set of health care claims, the method further comprising: denoising, by the one or more processors, the first set of health care claims to generate a second set of health care claims that is a subset of the first set of health care claims; wherein extracting the data set of medical entities comprises: extracting, by the one or more processors, the data set of medical entities from the clinical information contained in the second set of health care claims.

Example 5: the computer-implemented method of Examples 1-4, wherein determining the relationship strengths comprises: determining, by the one or more processors and for each of the pairs of the medical entities, a first conditional probability that a first entity in the pair is true given a second entity in the pair is true and a second conditional probability that the second entity in the pair is true given that the first entity in the pair is true.

Example 6: the computer-implemented method of Examples 1-4, wherein determining the relationship strengths between the pairs of the medical entities comprises: performing a Bayesian statistical analysis of the set of medical entities from the clinical information; and determining, by the one or more processors, the relationship strengths between the pairs of the medical entities based on the Bayesian statistical analysis.

Example 7: the computer-implemented method of Examples 1-6, wherein the medical entities comprise a diagnosis, a procedure, and a medication; and determining the relationship strengths comprises: determining, by the one or more processors, a first conditional probability that the diagnosis is true given the procedure is true; determining, by the one or more processors, a first conditional probability that the procedure is true given the diagnosis is true; determining, by the one or more processors, a second conditional probability that the diagnosis is true given the medication is true; determining, by the one or more processors, a first conditional probability that the medication is true given the diagnosis is true; determining, by the one or more processors, a second conditional probability that the procedure is true given the medication is true; and determining, by the one or more processors, a second conditional probability that the medication is true given the procedure is true.

Example 8: the computer-implemented method of Example 7, wherein extracting the set of medical entities from the clinical information further comprises: extracting, by the one or more processors, metadata from the clinical information that links the diagnosis to the procedure; and determining the first conditional probability that the diagnosis is true given the procedure is true comprises: determining, by the one or more processors, the first conditional probability that the diagnosis is true given the procedure is true based on the metadata.

Example 9: the computer-implemented method of Examples 7-8, wherein the computer-implemented method further comprises: determining, by the one or more processors, a subset of medications in the set of medical entities having a common proprietary name; determining, by the one or more processors, medical substance names associated with the subset of medications having the common proprietary name; and determining, by the one or more processors, one or more product National Drug Codes (NDCs) that each have at least of the medical substance names; wherein the medication is representative of all of the one or more medications corresponding to the one or more product NDCs.

Example 10: the computer-implemented method of Example 9, wherein determining the one or more product National Drug Codes (NDCs) comprises: accessing, by the one or more processors, a database that associates NDCs with medical substance names; wherein the database comprises a Center for Disease Control (CDC) and Prevention database or a Federal Drug Administration (FDA) database.

Example 11: the computer-implemented method of Examples 7-10, wherein the diagnosis comprises an International Classification of Diseases (ICD) code; the procedure comprises a Current Procedural Terminology (CPT) code or a Healthcare Common Procedure Coding System (HCPCS) code; and the medication comprises a National Drug Code (NDC).

Example 12: the computer-implemented method of Examples 1-11, wherein the computer-implemented method further comprises: validating, by the one or more processors, an inference output from a machine-learned model using the knowledge graph data structure.

Example 13: the computer-implemented method of Example 12, wherein the machine-learned model is configured to extract medical information from an electronic health record.

Example 14: the computer-implemented method of Example 12, wherein the machine-learned model is configured to recommend a medical code for clinical information contained in an electronic health record; wherein the medical code is an International Classification of Diseases (ICD) code, Current Procedural Terminology (CPT) code, or a Healthcare Common Procedure Coding System (HCPCS) code.

Example 15: a system comprises one or more processors; and one or more memories storing processor-executable instructions that, when executed by the one or more processors, cause the one or more processors to perform operations comprising: receiving a set of structured records containing clinical information associated with one or more patients; extracting a set of medical entities from the clinical information; determining relationship strengths between pairs of the medical entities; and configuring a computer-readable memory with a knowledge graph data structure in which nodes of the knowledge graph data structure represent the medical entities and the nodes are related to one another in the knowledge graph data structure based on the relationship strengths.

Example 16: the system of Example 15, wherein the medical entities comprise a diagnosis, a procedure, and a medication; and determining the relationship strengths comprises: determining a first conditional probability that the diagnosis is true given the procedure is true; determining a first conditional probability that the procedure is true given the diagnosis is true; determining a second conditional probability that the diagnosis is true given the medication is true; determining a first conditional probability that the medication is true given the diagnosis is true; determining a second conditional probability that the procedure is true given the medication is true; and determining a second conditional probability that the medication is true given the procedure is true.

Example 17: the system of Example 16, wherein the operations further comprise: determining a subset of medications in the set of medical entities having a common proprietary name; determining medical substance names associated with the subset of medications having the common proprietary name; determining one or more product National Drug Codes (NDCs) that each have at least of the medical substance names; wherein the medication is representative of all of the one or more medications corresponding to the one or more product NDCs.

Example 18: the system of Example 17, wherein determining the one or more product National Drug Codes (NDCs) comprises: accessing a database that associates NDCs with medical substance names; wherein the database comprises a Center for Disease Control (CDC) and Prevention database or a Federal Drug Administration (FDA) database.

Example 19: the system of Examples 15-18, wherein the operations further comprise: validating an inference output from a machine-learned model using the knowledge graph data structure.

Example 20: one or more non-transitory computer-readable media store processor-executable instructions that, when executed by one or more processors, cause the one or more processors to perform operations comprising: receiving a set of structured records containing clinical information associated with one or more patients; extracting a set of medical entities from the clinical information; determining relationship strengths between pairs of the medical entities; and configuring a computer-readable memory with a knowledge graph data structure in which nodes of the knowledge graph data structure represent the medical entities and the nodes are related to one another in the knowledge graph data structure based on the relationship strengths.

Throughout this specification, components, operations, or structures described as a single instance may be implemented as multiple instances. Although individual operations of one or more methods (or processes, techniques, routines, etc.) are illustrated and described as separate operations, two or more of the individual operations may be performed concurrently or otherwise in parallel, and nothing requires that the operations be performed in the order illustrated. Structures and functionality (e.g., operations, steps, blocks) presented as separate components in example configurations may be implemented as a combined structure, functionality, or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, operations, blocks, or instructions. These may constitute and/or be implemented by software (e.g., code embodied on a non-transitory, machine-readable medium), hardware, or a combination thereof. In hardware, the routines, etc., may represent tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware component that operates to perform certain operations as described herein.

In various embodiments, a hardware component may be implemented mechanically or electronically. For example, a hardware component may comprise dedicated circuitry or logic that is permanently configured (e.g., as a special-purpose processor, such as a field programmable gate array (FPGA) or an application-specific integrated circuit (ASIC)) to perform certain operations. A hardware component may also or instead comprise programmable logic or circuitry (e.g., as encompassed within one or more general-purpose processors and/or other programmable processor(s)) that is temporarily configured by software to perform certain operations.

Accordingly, the term “hardware component” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where the hardware components include a general-purpose processor configured using software, the general-purpose processor may be configured as respective different hardware components at different times. Software may accordingly configure a processor, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple of such hardware components exist contemporaneously, communications may be achieved through signal transmission (e.g., over appropriate circuits and buses) that connect the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information).

As noted above, the various operations of example methods (or processes, techniques, routines, etc.) described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions. The components referred to herein may, in some example embodiments, comprise processor-implemented components.

Moreover, each operation of processes illustrated as logical flow graphs may represent a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

The terms “coupled” and “connected,” along with their derivatives, may be used. In particular embodiments, “connected” may be used to indicate that two or more elements are in direct physical or electrical contact with each other, although the context in the description may dictate otherwise when it is apparent that two or more elements are not in direct physical or electrical contact. “Coupled” may mean that two or more elements are in direct physical or electrical contact. However, “coupled” may also mean that two or more elements are not in direct contact with each other, yet still co-operate, transmit between, or interact with each other.

An algorithm may be considered to be a self-consistent sequence of acts or operations leading to a desired result. These include physical manipulations of physical quantities. Usually, though not necessarily, these quantities take the form of electrical, magnetic, or optical signals capable of being stored, transferred, combined, compared, and otherwise manipulated. These signals are commonly referred to as bits, values, elements, symbols, characters, terms, numbers, flags, or the like. It should be understood, however, that all of these and similar terms are to be associated with the appropriate physical quantities and are merely convenient labels applied to these quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “some embodiments,” “one embodiment,” “an embodiment,” “in some examples,” or variations thereof means that a particular element, feature, structure, characteristic, operation, or the like described in connection with the embodiment is included in at least one embodiment, but not every embodiment necessarily includes the particular element, feature, structure, characteristic, operation, or the like. Different instances of such a reference in various places in the specification do not necessarily all refer to the same embodiment, although they may in some cases. Moreover, different instances of such a reference may describe elements, features, structures, characteristics, operations, or the like be combined in any manner as an embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless the context of use clearly indicates otherwise, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “set” is intended to mean a collection of elements and can be a null set (i.e., a set containing zero elements) or may comprise one, two, or more elements. A “subset” is intended to mean a collection of elements that are all elements of a set, but that does not include other elements of the set. A first subset of a set may comprise zero, one, or more elements that are also elements of a second subset of the set. The first subset may be said to be a subset of the second subset if all the elements of the first subset are elements of the second subset, while also being a subset of the set. However, if all the elements of the second subset are also elements of the first subset (in addition to all the elements of the first subset being elements of the second subset), the first subset and the second subset are a single subset/not distinct.

For the purposes of the present disclosure, the term “a” or “an” entity refers to one or more of that entity. As such, the terms “a” or “an”, “one or more”, and “at least one” can be used interchangeably herein unless explicitly contradicted by the specification using the word “only one” or similar. For example, “a first element” may functionally be interpreted as “a first one or more elements” or a “first at least one element.” Unless otherwise apparent from the context of use, reference in the present disclosure to a same set of “one or more processors” (or a same “plurality of processors,” etc.) performing multiple operations can encompass implementations in which performance of the operations is divided among the processor(s) in any suitable way. For example, “generating, by one or more processors, X; and generating, by the one or more processors, Y” can encompass: (1) implementations in which a first subset of the processors (e.g., in a first computing device) generates X and an entirely distinct, second subset of the processors (e.g., in a different, second computing device) independently generates Y; (2) implementations in which one or more or all of the processor(s) (e.g., one or multiple processors in the same device, or multiple processors distributed among multiple devices) contribute to the generation of X and/or Y; and (3) other variations. This may similarly be applied to any other component or feature similarly recited (e.g., as “a component”, “a feature”, “one or more components”, “one or more features”, “a plurality of components”, “a plurality of features”). Moreover, the performance of certain of the operations may be distributed among the one or more components, not only residing within a single machine, but deployed across a number of machines. The set of components may be located in a single geographic location (e.g., within a home environment, an office environment, a cloud environment). In other example embodiments, the set of components may be distributed across two or more geographic locations. Further, “a machine-learned model”, equivalent terms (e.g., “machine learning model,” “machine-learning model,” “machine-learned component”, “artificial intelligence”, “artificial intelligence component”), or species thereof (e.g., “a large language model”, “a neural network”) may include a single machine-learned model or multiple machine-learned models, such as a pipeline comprising two or more machine-learned models arranged in series and/or parallel, an agentic framework of machine-learned models, or the like.

An “artificial intelligence” or “artificial intelligence component” may comprise a machine-learned model. A machine-learned model may comprise a hardware and/or software architecture having structural hyperparameters defining the model's architecture and/or one or more parameters (e.g., coefficient(s), weight(s), biase(s), activation function(s) and/or action function type(s) in examples where the activation function and/or function type is determined as part of training, clustering centroid(s)/medoid(s), partition(s), number of trees, tree depth, split parameters) determined as a result of training the machine-learned model based at least in part on training hyperparameters (e.g., for supervised, semi-supervised, and reinforcement learning models) and/or by iteratively operating the machine-learned model according to the training hyperparameters (e.g., for unsupervised machine-learned models).

In some examples, structural hyperparameter(s) may define component(s) of the model's architecture and/or their configuration/order, such as, for example, the configuration/order specifying which input(s) are provided to one component and which output(s) of that component are provided as input to other component(s) of the machine-learned model; a number, type, and/or configuration of component(s) per layer; a number of layers of the model; a number and/or type of input nodes in an input layer of the model; a number and/or type of nodes in a layer; a number and/or type of output nodes of an output layer of the model; component dimension (e.g., input size versus output size); a number of trees; a maximum tree depth; node split parameters; minimum number of samples in a leaf node of a tree; and/or the like. The component(s) of the model may comprise one or more activation functions and/or activation function type(s) (e.g., gated linear unit (GLU), such as a rectified linear unit (ReLU), leaky RELU, Gaussian error linear unit (GELU), Swish, hyperbolic tangent), one or more attention mechanism and/or attention mechanism types (e.g., self-attention, cross-attention), nodes and split indications and/or probabilities in a decision tree, and/or various other component(s) (e.g., adding and/or normalization layer, pooling layer, filter). Various combinations of any these components (as defined by the structural hyperparameter(s)) may result in different types of model architectures, such as a transformer-based machine-learned model (e.g., encoder-only model(s), encoder-decoder model(s), decoder-only models, generative pre-trained transformer(s) (GPT(s))), neural network(s), multi-layer perceptron(s), Kolmogorov-Arnold network(s), clustering algorithm(s), support vector machine(s), gradient boosting machine(s), and/or the like. The structural parameters and components a machine-learned model comprises may vary depending on the type of machine-learned model.

Training hyperparameter(s) may be used as part of training or otherwise determining the machine-learned model. In some examples, the training hyperparameter(s), in addition to the training data and/or input data, may affect determining the parameter(s) of the target machine-learned model. Using a different set of training hyperparameters to train two machine-learned models that have the same architecture (i.e., the same structural hyperparameters) and using the same training data may result in the parameters of the first machine-learned model differing from the parameters of the second machine-learned model. Despite having the same architecture and having been trained using the same training data, such machine-learned models may generate different outputs from each other, given the same input data. Accordingly, accuracy, precision, recall, and/or bias may vary between such machine-learned models.

In some examples, training hyperparameter(s) may include a train-test split ratio, activation function and/or activation function type (e.g., in examples like Kolmogorov-Arnold networks (KANs) where the activation function type is determined as part of training from an available set of activation functions and/or limits on the activation function parameters specified by the training hyperparameters), training stage(s) (e.g., using a first set of hyperparameters for a first epoch of training, a second set of hyperparameters for a second epoch of training), a batch size and/or number of batches of data in a training epoch, a number of epochs of training, the loss function used (e.g., L1, L2, Huber, Cauchy, cross entropy), the component(s) of the machine-learned model that are altered using the loss for a particular batch or during a particular epoch of training (e.g., some components may be “frozen,” meaning their parameters are not altered based on the loss), learning rate, learning rate optimization algorithm type (e.g., gradient descent, adaptive, stochastic) used to determine an alteration to one or more parameters of one or more components of the machine-learned model to reduce the loss determined by the loss function, learning rate scheduling, and/or the like.

In some examples, the structural hyperparameters and/or the training hyperparameters may be determined by a hyperparameter optimization algorithm or based on user input, such as a software component written by a user or generated by a machine-learned model. The machine-learned model may include any type of model configured, trained, and/or the like to generate a prediction output for a model input. In some examples, any of the logic, component(s), routines, and/or the like discussed herein may be implemented as a machine-learned model.

The machine-learned model may include one or more of any type of machine-learned model including one or more supervised, unsupervised, semi-supervised, and/or reinforcement learning models. Training a machine-learned model may comprise altering one or more parameters of the machine-learned model (e.g., using a loss optimization algorithm) to reduce a loss. Depending on whether the machine-learned model is supervised, semi-supervised, unsupervised, etc. this loss may be determined based at least in part on a difference between an output generated by the model and ground truth data (e.g., a label, an indication of an outcome that resulted from a system using the output), a cost function, a fit of the parameter(s) to a set of data, a fit of an output to a set of data, and/or the like. In some examples, determining an output by a machine-learned model may comprise executing a set of inference operations executed by the machine-learned model according to the target machine-learned model's parameter(s) and structural hyperparameter(s) and using/operating on a set of input data.

Moreover, any discussion of receiving data associated with an individual that may be protected, confidential, or otherwise sensitive information, is understood to have been preceded by transmitting a notice of use of the data to a computing device, account, or other identifier (collectively, “identifier”) associated with the individual, receiving an indication of authorization to use the data from the identifier, and/or providing a mechanism by which a user may cause use of the data to cease or a copy of the data to be provided to the user.

Upon reading this disclosure, those of skill in the art will appreciate still additional alternative structural and functional designs through the principles disclosed herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The patent claims at the end of this patent application are not intended to be construed under 35 U.S.C. § 112(f) unless traditional means-plus-function language is expressly recited, such as “means for” or “step for” language being explicitly recited in the claim(s).

The embodiments of the present disclosure have been presented for purposes of illustration and description, but are not intended to be exhaustive or limited to the disclosure in the form disclosed. Many modifications and variations may be apparent to those of ordinary skill in the art without departing from the scope and spirit of the disclosure. The aspects of the disclosure herein were chosen and described to best explain the principles of the disclosure and the practical application thereof, and to enable others of ordinary skill in the art to understand the disclosure with various modifications as are suited to the particular use contemplated.

1. A computer-implemented method, comprising:
receiving, by one or more processors, a set of structured records containing clinical information associated with one or more patients;
extracting, by the one or more processors, a set of medical entities from the clinical information;
determining, by the one or more processors, relationship strengths between pairs of the medical entities; and
configuring, by the one or more processors, a computer-readable memory with a knowledge graph data structure in which nodes of the knowledge graph data structure represent the medical entities and the nodes are related to one another in the knowledge graph data structure based on the relationship strengths; and
validating, by the one or more processors, an inference output from a machine-learned model using the knowledge graph data structure.

2. The computer-implemented method of claim 1, wherein the set of structured records comprises a set of health care claims.

3. The computer-implemented method of claim 2, wherein the set of health care claims comprises a provider claim and a pharmacy claim.

4. The computer-implemented method of claim 2, wherein the set of health care claims is a first set of health care claims, the method further comprising:
denoising, by the one or more processors, the first set of health care claims to generate a second set of health care claims that is a subset of the first set of health care claims;
wherein extracting the data set of medical entities comprises:
extracting, by the one or more processors, the data set of medical entities from the clinical information contained in the second set of health care claims.

5. The computer-implemented method of claim 1, wherein determining the relationship strengths comprises:
determining, by the one or more processors and for each of the pairs of the medical entities, a first conditional probability that a first entity in the pair is true given a second entity in the pair is true and a second conditional probability that the second entity in the pair is true given that the first entity in the pair is true.

6. The computer-implemented method of claim 1, wherein determining the relationship strengths between the pairs of the medical entities comprises:
performing a Bayesian statistical analysis of the set of medical entities from the clinical information; and
determining, by the one or more processors, the relationship strengths between the pairs of the medical entities based on the Bayesian statistical analysis.

7. The computer-implemented method of claim 1, wherein the medical entities comprise a diagnosis, a procedure, and a medication; and
wherein determining the relationship strengths comprises:
determining, by the one or more processors, a first conditional probability that the diagnosis is true given the procedure is true;
determining, by the one or more processors, a first conditional probability that the procedure is true given the diagnosis is true;
determining, by the one or more processors, a second conditional probability that the diagnosis is true given the medication is true;
determining, by the one or more processors, a first conditional probability that the medication is true given the diagnosis is true;
determining, by the one or more processors, a second conditional probability that the procedure is true given the medication is true; and
determining, by the one or more processors, a second conditional probability that the medication is true given the procedure is true.

8. The computer-implemented method of claim 7, wherein extracting the set of medical entities from the clinical information further comprises:
extracting, by the one or more processors, metadata from the clinical information that links the diagnosis to the procedure; and
wherein determining the first conditional probability that the diagnosis is true given the procedure is true comprises:
determining, by the one or more processors, the first conditional probability that the diagnosis is true given the procedure is true based on the metadata.

9. The computer-implemented method of claim 7, further comprising:
determining, by the one or more processors, a subset of medications in the set of medical entities having a common proprietary name;
determining, by the one or more processors, medical substance names associated with the subset of medications having the common proprietary name; and
determining, by the one or more processors, one or more product National Drug Codes (NDCs) that each have at least of the medical substance names;
wherein the medication is representative of all of the one or more medications corresponding to the one or more product NDCs.

10. The computer-implemented method of claim 9, wherein determining the one or more product National Drug Codes (NDCs) comprises:
accessing, by the one or more processors, a database that associates NDCs with medical substance names;
wherein the database comprises a Center for Disease Control (CDC) and Prevention database or a Federal Drug Administration (FDA) database.

11. The computer-implemented method of claim 7, wherein the diagnosis comprises an International Classification of Diseases (ICD) code;
wherein the procedure comprises a Current Procedural Terminology (CPT) code or a Healthcare Common Procedure Coding System (HCPCS) code; and
wherein the medication comprises a National Drug Code (NDC).

12. (canceled)

13. The computer-implemented method of claim 1, wherein the machine-learned model is configured to extract medical information from an electronic health record.

14. The computer-implemented method of claim 1, wherein the machine-learned model is configured to recommend a medical code for clinical information contained in an electronic health record;
wherein the medical code is an International Classification of Diseases (ICD) code, Current Procedural Terminology (CPT) code, or a Healthcare Common Procedure Coding System (HCPCS) code.

15. A system, comprising:
one or more processors; and
one or more non-transitory computer-readable memories storing processor-executable instructions that, when executed by the one or more processors, cause the one or more processors to perform operations comprising:
receiving a set of structured records containing clinical information associated with one or more patients;
extracting a set of medical entities from the clinical information;
determining relationship strengths between pairs of the medical entities; and
configuring a computer-readable memory with a knowledge graph data structure in which nodes of the knowledge graph data structure represent the medical entities and the nodes are related to one another in the knowledge graph data structure based on the relationship strengths; and
validating an inference output from a machine-learned model using the knowledge graph data structure.

16. The system of claim 15, wherein the medical entities comprise a diagnosis, a procedure, and a medication; and
wherein determining the relationship strengths comprises:
determining a first conditional probability that the diagnosis is true given the procedure is true;
determining a first conditional probability that the procedure is true given the diagnosis is true;
determining a second conditional probability that the diagnosis is true given the medication is true;
determining a first conditional probability that the medication is true given the diagnosis is true;
determining a second conditional probability that the procedure is true given the medication is true; and
determining a second conditional probability that the medication is true given the procedure is true.

17. The system of claim 16, wherein the operations further comprise:
determining a subset of medications in the set of medical entities having a common proprietary name;
determining medical substance names associated with the subset of medications having the common proprietary name; and
determining one or more product National Drug Codes (NDCs) that each have at least of the medical substance names;
wherein the medication is representative of all of the one or more medications corresponding to the one or more product NDCs.

18. The system of claim 17, wherein determining the one or more product National Drug Codes (NDCs) comprises:
accessing a database that associates NDCs with medical substance names;
wherein the database comprises a Center for Disease Control (CDC) and Prevention database or a Federal Drug Administration (FDA) database.

19. (canceled)

20. One or more non-transitory computer-readable media storing processor-executable instructions that, when executed by one or more processors, cause the one or more processors to perform operations comprising:
receiving a set of structured records containing clinical information associated with one or more patients;
extracting a set of medical entities from the clinical information;
determining relationship strengths between pairs of the medical entities; and
configuring a computer-readable memory with a knowledge graph data structure in which nodes of the knowledge graph data structure represent the medical entities and the nodes are related to one another in the knowledge graph data structure based on the relationship strengths; and
validating an inference output from a machine-learned model using the knowledge graph data structure.

21. The system of claim 15, wherein determining the relationship strengths between the pairs of the medical entities comprises:
performing a Bayesian statistical analysis of the set of medical entities from the clinical information; and
determining the relationship strengths between the pairs of the medical entities based on the Bayesian statistical analysis.

22. The system of claim 15, wherein the machine-learned model is configured to extract medical information from an electronic health record.
